(19) United States
(12) Patent Application Publication
(10) Pub. No.: US 2008/0234668 A1
(43) Pub. Date: Sep. 25, 2008

(54) Method and Device For the Macular Degeneration Treatment

(75) Inventors: Leonid Andriyovych Linnik, Odessa (UA); Olexander Stanislavovych Pekaryk, Kyiv (UA)

Correspondence Address: OGILVY RENAULT LLP, 1981 MCGILL COLLEGE AVENUE, SUITE 1600, MONTREAL, QC, H3A2Y3, CA

(73) Assignee: VISION AID INC., OakBank, MB (CA)

(21) Appl. No.: 12/065,442

(22) PCT Filed: Dec. 14, 2005
(86) PCT No.: PCT/UA2005/000057

§ 371 Date: Feb. 29, 2008

(30) Foreign Application Priority Data
Aug. 31, 2005 (UA) A 2005 08464

(51) Int. Cl. A61F 9/008

(52) U.S. Cl. 606/4

(57) ABSTRACT

A method of the macula degeneration treatment by irradiating of the retina damaged areas through the pupil wherein the technique of the irradiation performed by the quasi-monochromatic light pulse in visible and near infra-red spectral range with an energy level that does not exceed 10⁻⁵ joules.

### TECHNICAL FIELD

The present invention relates to a device and a method for medicine and concerns ophthalmology. This invention can be used for the treatment of retina degeneration diseases, particularly for macular degeneration treatment.

### BACKGROUND AND PRIOR ART

The method of macular degeneration (MD) treatment by low-energy X-ray radiation is known. Radiotherapy system TheraSight Ocular Brachytherapy System, suggested by Theragenics Corporation, for implementation of this method is on the stage of clinical trials and there are no certain data available concerning the application results. (“Radiotherapy in age-related macular degeneration”, Gripp, S.; Stammen, J.; Petersen, C; Hartmann, A.; Willers, R.; Althaus, C, Int. J. Radiat. Oncol. Biol. Phys. 2002 Feb. 1; 52 (2): 489-95).

The other known method of macular degeneration treatment is transpupillary thermotherapy (TTT). TTT is a technique in which heat is delivered to the choroids and retinal pigment epithelium through the pupil using a diode laser in combination with drug therapy. This method was developed and introduced by IRIDEX Company. The method employs a special device which includes an infrared CW laser. This method was developed for the “wet” or neovascular form of MD treatment. Presently no information exists concerning applicability of the TTT method for the treatment of “dry” or non-neovascular MD. (“Transpupillary thermotherapy of juxtafoveal recurrent choroidal neovascularization” Cardillo-Piccolino, -F; Eandi, -C-M; Ventre, -L; Rigault-De-La-Longrais, -R-C; Grignolo, -F-M, Eur-J-Opthalmol. 2003 June; 13(5): 453-60; web site of IRIDEX company—www.iredex.com).

A drawback of the above mentioned method is the use of substantial radiation power to ensure necessary thermal action that increases the risk of unforeseen permanent damage of the eye tissue. Though the energy values at the bottom of the eye are less than threshold level, there is real danger for thermal injury of the eye tissues. In the proposed method and device energy, the level is much lower than the threshold level and thus the probability of thermal injury of the eye tissue are significantly reduced.

The most relevant prior art related to the present invention by the physical principles and medical effect is the method of photodynamic therapy (PDT) in the so called “wet” form of MD. In this treatment, the retina of the eye undergoes irradiation by laser radiation with an energy level that is lower than the energy needed for the coagulation of retina tissue. Under these conditions, the changes initiated by light take place within the eye tissue at the biochemical level of cell function. In the PDT method the use chemical optical sensitizer (Visudyne, Novartis) is indispensable for increasing light efficiency.

An optical sensitizer selectively magnifies absorption of laser light by eye tissue. There are special types of optical sensitizers for each laser used in the process. Visudyne is delivered to the vessels of the retina by intravenous injection. Using Visudyne, 16% of the patients were noted to show improvement in visual acuity (VA); this is almost twice as much relative to that in those who did not undergo PTD with Visudyne. The VISULASTM 690s system by the Carl Zeiss Meditiec Company can be considered as the most relevant art.

The energy level that affects eye tissues by using the above method remains unfortunately high. Furthermore, the method requires the injection of the optical sensitizer in the patient's bloodstream and this has a toxic impact upon the human organism as a whole. (Novartis—http://www.novartis.com/; Carl Zeiss Meditec AG—http://www.meditec.zeiss.com; Booklets of Carl Zeiss Meditec AG; “Photodynamic therapy increases the eligibility for feeder vessel treatment of choroidal neovascularization caused by age-related macular degeneration.”, Piermarocchi, S.; Lo-Giudice, G.; Sartore, M.; Friede, F.; Segato, T.; Pilotto, E.; Midena, E.; Am. J. Opthalmol. 2002 April; 133(4): 572-5).

Moreover, all previously mentioned treatment methods and devices require direct action on the eye tissue. In other words they do not affect the cause of the disease, but rather the symptoms. Macular degeneration is known to be a disease concerned with the immunodeficiency state of the human.

One of the VISULASTM 690s disadvantages is the necessity to use it in combination with a special chemical agent that is toxic and acts as optical sensitizer. Use of these substances on patients requires special conditions which can only be effectively administered in a hospital environment.

Another disadvantage of this system is the technical complexity associated with the requirements regarding energy density uniformity of irradiation over the affected eye areas. This problem poses stringent requirements for the beam forming system and beam control system. The significant technical problem is the laser power control and laser energy stabilization. Furthermore, high coherence of laser radiation predetermines certain difficulties in forming a uniform light field over the affected parts of the retina to be irradiated.

Technical complexity and the special requirements for the operating conditions result in additional professional requirements for medical staff and as a consequence, higher cost for service and equipment.

The most common shortcoming of existing systems is that they are designed for the treatment of “wet”, neovascular macular degeneration. For the “dry” form of macular degeneration such systems are not effective.

A common significant disadvantage of the noted devices and methods, including, VISULASTM 690s system, is that they provide symptomatic treatment of the systemic disease without the impact on cause of the onset of symptoms.

The proposed method and device according to one embodiment of the invention results in stimulation of the immune system, resulting in a positive effect on the eye vascular system.

### DESCRIPTION OF THE INVENTION

A treatment method of macular degeneration including age-related macular degeneration (AMD) according to the present invention, envisages using the special device in a prescribed manner for transpupillar irradiation. Affected areas of the patient's retina are irradiated with visible and near infrared quasi-monochromatic light pulses having energy values that do not result in coagulation of irradiated retina tissue. The correspondent average radiation energy does not exceed 10⁻⁵ Joules per pulse.

One object of one embodiment of the present invention is to provide a method of treating macula degeneration, comprising irradiating damaged retinal areas through the pupil, wherein irradiation is conducted using quasi-monochromatic light pulse in the visible and near infra-red spectral range with an energy and that does not exceed 10⁻⁵ joules.

A further object of one embodiment of the present invention is to provide a device for macula degeneration treatment, having a source of radiation, radiation directional pattern forming means, means to deliver the radiation to the given part of the retina through the pupil, power supply means, indicator and control means; said device comprising at least one light emitting diode (LED) employed as the source of radiation.

The procedure mentioned above may be performed without any chemicals or medical substances being injected into the patient.

The main distinction of the proposed device is the use of LEDs (light-emitting diodes) as the source of radiation. They possess proper characteristics:
High radiation power (0.01-0.25 microwatt);
High light output efficiency;
Narrow radiation spectral band (half-width up to 10 nm);
Low current demand compared to a laser (<0.25 milliampere);
Quasi-linear relation between power of radiation and current supply;
Small size and weight with simplified spatial configuration alteration;
Low cost; and
High market availability for a wide range of LEDs which emit in the different bands of visible and near infra-red spectral range.

The device according to one embodiment of the invention is a programmed and controlled LED source of quasi-monochromatic radiation, which preferably comprises one or more arrays of ultra bright LEDs operating in the visible and infra-red spectral ranges. The use of LED instead of lasers allows for increasing stability and controllability of the light source due to the dependence of LED brightness on the current supplied. This enables the formation of light pulses with stable, reproducible, parameters such as energy, duration, shape, repetition rate, all of which may be established in advance. As a corollary to these features, reliability increases and, overall dimensions and weight decrease. The LEDs by themselves in contrast to lasers do not require special technical servicing. Further, contemporary LEDs are commercially available and irradiate in the different bands of visible and near infra-red ranges and cover these ranges almost entirely. This is distinct from lasers which emit at fixed wavelengths. The low coherence of LED radiation simplifies the problem of uniform light field formation at those parts of the retina which must be irradiated (in particular, speckle does not occur). The required uniformity may be achieved by an LED array in an appropriate configuration. Additionally, in some cases diffusers may be used as light homogenizers. As an example opal glass may be used.

Unlike lasers, LEDs have small dimensions and concomitant weight, low energy consumption and simplicity of use. This results in a device that is easy-to-use, with a weight of approximately 300-400 g. An alignment system is also provided (special glasses, holders, racks), as well as a radiation delivery system.

The device and the treatment method are simple and do not require special technical training of medical staff or availability of specific conditions for the procedures. All parameters of the procedure algorithm, including the duration of irradiation, are controlled by software. The medical professional need only choose an algorithm for each case. The maintenance costs of the method and the device for macular degeneration treatment is lower in comparison with competitive methods and devices.

The method and the device are most effective for the “dry”, non-neovascular form of MD treatment or visual function stabilization (loss of visual acuity more than 50%) and for the early stages of “wet”, neovascular form of macular degeneration treatment.

Regeneration, stabilization and improvement of visual function coincide with experimental data concerning a 15-20% increase in DNA content in the retina cell nucleolus and with appreciable positive human immune system response to the retina irradiation by the monochromatic light. Consequently, it is contended that the influence is very directed on the reasons of the disease origin lied in the age-specific or artificial immunity suppression.

There is a list of main symptomatic characteristics by which one can diagnose macular degeneration. When using the proposed method and device, significant positive changes in visual functions are realized. The list of symptoms and changes is tabulated in Table 1.

Visual function stimulation, its stabilization and improvement with the presence of MD occur owing to the specific quasi-monochromatic, low-intensity light pulse with selected wavelength action. The results of experimental studies have shown that the main reason for visual function stimulation is positive immune system response to the retina irradiation by the monochromatic light. In other words, the immune system is stimulated at first, and then as a consequence, the eye vascular system is improved which, in turn, leads to visual function stabilization and regeneration. Radiation with the different wavelengths effects the specific response of the immune system. Every radiation wavelength has an impact on the immune system (quantitative and qualitative). Impact effectiveness depends upon the spectral characteristics of the radiation source as well as upon mode of operation. The mode of operation, is referring to irradiation duration, quantity of the irradiation sessions, irradiation energy, light pulse repetition rate, light pulse shape, and quantity of light pulses. All these characteristics in combination provide for the most effective response of the immune system. The device according to the present invention allows each mode for the MD treatment and eye visual function stimulation.

### BRIEF DESCRIPTION OF THE DRAWINGS

FIG. 1 is a flow chart for MD treatment.

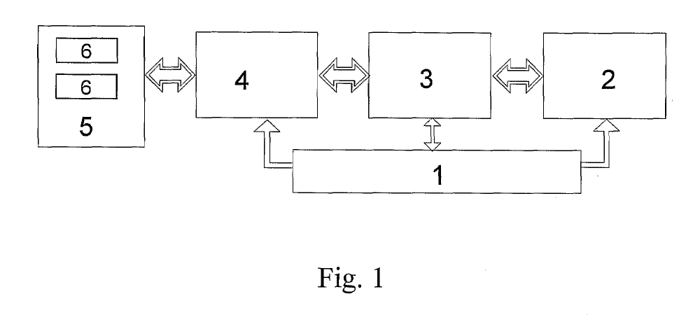

### BEST MODE FOR CARRYING OUT THE INVENTION

The device consists of a power supply unit 1, indicator and control unit 2, controller 3, LEDs control unit 4, and unit 5

The power supply unit 1 is the storage battery. It has a corresponding charger. The charger is electrically connected to the indicator and control unit 2, controller 3, and LEDs control unit 4.

Indicator and control unit 2 is used by an operator (not shown) for the device operation mode selection and for the indication of running status of the device. Specifically, it can display the data concerning all units readiness for operation, error messages and prompts for the operator. Indicator and control unit 2 is electrically connected to the controller 3.

Controller 3 performs device operation control according to the programs that determine operational algorithm of the device. Also controller 3 performs diode calibration, monitoring of device operation, emergency protection, recording and storage of information concerning date, duration and operating mode of the device. Controller 3 ensures access authorization to the device control. Controller 3 is electrically connected to the indictor and control unit 2 and LEDs control unit 4.

LEDs control unit 4 transforms the controller commands into current supply pulses of light emitting diodes. Parameters of the current supply pulses correspond to the given control program for the operating mode. LEDs control unit 4 is electrically connected to the LEDs assembled on the printed circuit board for example as an arrays 6, which are integrated in irradiating light unit 5.

The irradiating light unit 5 forms directional patterns of irradiating light and delivers light to one or two eyes of the patient (not shown) at once. The unit 5 allows for adjustment of the arrays 6 spatial configuration to provide irradiating light delivered in the most effective way to both eyes at once. The unit 5 may be used in a stationary position or may be held and adjusted manually by the patient. Housings of LED arrays 6 may be sealed. The unit 5 box is suitable for the disinfection.

After switching on the power supply that is independent storage battery operated, information concerning battery charging condition and power supply switching on indication appears on the display of control and indication unit 2.

Subsequently, a system self-test occurs. When the indication “calibration” is on, calibration of the light sources is performed.

From the self test result, the indication of the ready state condition of the device (“Ready” or “Failure”) appears.

When this procedure is completed the irradiating light unit 5 is optimally arranged with the respect to the patient's eyes.

The appropriate radiation mode is switched on (separate buttons “Mode 1”, “Mode 2” etc.) and irradiation begins (separate bottom “Start”).

Irradiation occurs according to the operating mode programs previously stored to the controller 3. In compliance with these programs, controller 3 manages the following parameters: exposure duration, light pulses amplitude, light pulses duration, light pulse repetition rate, and sequence of the LED radiation with different wavelengths.

Subject to the control programs the device can be operated in pulse or continuous wave mode of radiation.

The current supply can vary over the range of 0-40 mA with 1 mA steps. The exposure period can vary over the range of 0-15 minutes with 1 minute steps and the light pulse duration can vary over the range from 10 microseconds to 1 second; light pulse-repetition rate can vary over the range from 0 to 10 kHz.

According to test data, the optimum performance for the “dry” macular degeneration treatment is the course consisting of 10 sessions of 5 minutes each being held on out-patient basis three times per year. The arrays with LEDs emitting in green (520 nm) and infra-red (940 nm) spectral areas is used in this treatment protocol.

The irradiation parameters are as follows: light pulse repetition rate is 30 Hz, light pulses duration is 10 ms, exposition equals to 5 min, monochromatic radiation energy lies within the range 10⁻⁶÷10⁻⁵ joules. These energy values are subthreshold, i.e they do not cause coagulation of the irradiated retina tissues. The energy value assorts particularly within the above mentioned range depending on the pigmentation level, refraction and location of the degeneration area.

Treatment effect that consists in improvement of visual function (increase in visual acuity, lessening of central flows in the fields of vision and diminution of its density) became apparent on the next day. Immune response of the human organism becomes evident since the first instant after the irradiation and continue changing during the period of 14 days. For example, immunoregulatory index rises several times (up to 8÷10). Clinical effect remains during 3÷4 months.

TABLE 1

| Diagnostic characteristics | Results after treatment |
|---|---|
| Lines and objects contortion | Lessening and disappearance of contor- |
| | tion |
| Loss of visual acuity | After the first course 88% of patients |
| | have 26-30% increase in the visual |
| | acuity; after the second course 22- |
| | 25% more; |
| Disorganization of retina | Normalization of the retina reflexes |
| reflexes in the central area | in macula; |
| (macula); | |
| Limitation of the central visual | Decrease of the absolute scotomas in |
| field, presence of absolute and | the area and transfer fair quantity |
| relative scotomas; | of them into the relative scotomas. |
| | Relative scotomas disperse and vanish. |
| Disturbance of color perception. | Increase in cone cell light sensi- |
| | tivity by 33% (after second course |
| | by 40%) |
| Critical margining of blinking in | Increase by 95-101% |
| green and red colors. | |
| Schirmer's phenomenon | Increase by 207-230% |
| Disorder in lysosomal system of | Membrane-stabilizing effect and lyso- |
| pigment epithelium of retina and | some membrane functions increase, |
| blood microcirculation in | positive impact on the metabolic |
| choriocapillaris that feeds first | process in the cells of pigment |
| neuron structure. | epithelium and choriocapillaris. |

1. A method of treating macula degeneration comprising irradiating damaged retinal areas through the pupil, wherein irradiation is conducted using quasi-monochromatic light pulse in the visible and near infra-red spectral range with an energy, and that does not exceed 10⁻⁵ joules.

2. The method according to claim 1, wherein the irradiation is performed according to a computer program given in advance with at least one of synchronous or consecutive irradiation by quasi-monochromatic light in the spectral ranges, with the different pulse duration, different pulse repetition rate, different exposure periods or pulse number, different pulses shape and their relative time delay.

3. The method according to claim 1, wherein the irradiation is performed in combination with medical drugs.

4. The method according to claim 2, wherein the irradiation is performed in combination with medical drugs.

5. A device for macula degeneration treatment, having a source of radiation, radiation directional pattern forming means, means to deliver the radiation to the given part of the retina through the pupil, power supply means, indicator and control means; said device comprising at least one light emitting diode (LED) employed as the source of radiation.

6. The device according to claim 5, wherein said LED comprises at least one array of LEDs.

7. The device according to claim 6, wherein said array of LEDs comprises at least two groups of LEDs for radiating in two different spectral bands.

8. The device according to claim 5, further including a programmed controller and LED control unit, said controller being electrically connected to said indicator and control unit and to the LED control unit that forms current pulses running through the light emitting diodes.

9. The device according to claim 6, further including a programmed controller and LED control unit, said controller being electrically connected to said indicator and control unit and to the LED control unit that forms current pulses running through the light emitting diodes.

10. The device according to claim 7, further including a programmed controller and LED control unit, said controller being electrically connected to said indicator and control unit and to the LED control unit that forms current pulses running through the light emitting diodes.

11. The device according to claim 8, wherein said controller has an interface for programming and functioning according to said program, parameter change which provides for the controller commands being sent to the LED control unit to provide synchronous or consecutive irradiation by quasi-monochromatic light, with different pulse duration, different pulse repetition rate, different exposure period or pulse quantity, different pulse shape and their relative time delay.

12. The device according to claim 5, wherein said LEDs are arranged in the form of a polygon or oval.

13. The device according to claim 6, wherein said LEDs are arranged in the form of a polygon or oval.

14. The device according to claim 7, wherein said LEDs are arranged in the form of a polygon or oval.

15. The device according to claim 8, wherein said LEDs are arranged in the form of a polygon or oval.

16. The device according to claim 9, wherein said LEDs are arranged in the form of a polygon or oval.

17. The device according to claim 5, wherein said device for radiation directional pattern forming means comprises a radiation diffuser.

18. The device according to claim 6, wherein said device for radiation directional pattern forming means comprises a radiation diffuser.

19. The device according to claim 7, wherein said device for radiation directional pattern forming means comprises a radiation diffuser.

20. The device according to claim 8, wherein said device for radiation directional pattern forming means comprises a radiation diffuser.

21. The device according to claim 9, wherein said device for radiation directional pattern forming means comprises a radiation diffuser.

22. The device according to claim 10, wherein said device for radiation directional pattern forming means comprises a radiation diffuser.

23. The device according to claim 5, wherein the radiation diffuser comprises opal glass.

24. The device according to claim 6, wherein the radiation diffuser comprises opal glass.

25. The device according to claim 7, wherein the radiation diffuser comprises opal glass.

26. The device according to claim 8, wherein the radiation diffuser comprises opal glass.

27. The device according to claim 9, wherein the radiation diffuser comprises opal glass.

28. The device according to claim 10, wherein the radiation diffuser comprises opal glass.

29. The device according to claim 11, wherein the radiation diffuser comprises opal glass.

30. The device according to claim 17, wherein the radiation diffuser comprises opal glass.
